(19) United States
(12) Patent Application Publication
(10) Pub. No.: US 2026/0108385 A1
(43) Pub. Date: Apr. 23, 2026

(54) DEVICE FOR WETTING THE EYE AND METHOD THEREFOR

(71) Applicant: EyeNeb GmbH, Herrsching (DE)

(72) Inventors: Frank Bartels, Hattingen (DE); Juergen Rawert, Cologne (DE)

(21) Appl. No.: 18/987,140

(22) PCT Filed: Jun. 27, 2023
(86) PCT No.: PCT/EP2023/067409

§ 371 Date: Dec. 19, 2024

(30) Foreign Application Priority Data
Jun. 29, 2022 (DE) 10 2022 116 282.6

(51) Int. Cl. A61F 9/00

(52) U.S. Cl. CPC A61F 9/0008

(57) ABSTRACT

The device (1) for wetting the eye (2) with a liquid (F) comprises a reservoir (3) for storage of the liquid (F), a dispensing device (4) which is feedable from said reservoir (3) and which is orientable to the eye (2), and a triggering device (5) which activates same. The device (1) comprises a means for sensing (6) of a suitable position of the dispensing device (4) in relation to the eye (2) for wetting. The method for wetting the eye (2) with a liquid (F) comprises the step of continuously detecting the position of a dispensing device (4) designed to withdraw a liquid (F) from a reservoir (3) and to use it for wetting the eye (2). A means for sensing (6) transmits a signal when a suitable position of the dispensing device (4) in relation to the eye (2) for wetting has been sensed.

### FIELD OF THE INVENTION

The invention relates to the field of wetting an eye of a subject with a liquid. In particular, the invention relates to a device and a method for wetting an eye of a subject with a liquid containing a medicament, preferably for safe self-use by a user.

### PRIOR ART AND DISADVANTAGES

Devices for wetting the eyes with a liquid, in particular with a liquid containing a medicament, are known from the prior art. They are used to merely moisten a dry eye in a simple case, but also to treat different eye disorders.

U.S. Pat. No. 10,154,923 B2 describes a device and a method for wetting the eye, wherein the device conveys liquid from a reservoir to a vibrating plate at which atomization occurs. As a result of manual positioning of the device in front of the eye of the user, said eye is exposed to the mist. A disadvantage of this solution is the effect of the different distances chosen in repeated uses, even by the same user, which means different amounts of liquid reaching the eye and thus uneven treatment.

WO 2007/132 446 A2 discloses a device which comprises an eye cover into which fine droplets of liquid are delivered by means of an atomizer. Although in this case the distance between atomizer and eye is substantially constant, a comparatively large amount of mist is required in order to flood the interior of the eye cover, which is disadvantageous especially in respect of frequently high-priced medicaments.

U.S. Pat. No. 10,624,781 B2 proposes a device which is attached within or to the earpiece of conventional eyeglasses. Prior to wetting, a thin conduit comprising a nozzle must be manually slid into the field of view in front of the eye to be treated, and so the distance from the eye is substantially always the same. However, a disadvantage of this solution is the need for the presence of appropriate eyeglasses; moreover, the sliding of the conduit may be a challenge for older users.

EP 3 720 397 A1 discloses a wetting device comprising an auxiliary device which is used to set the correct distance and angle between the dispensing nozzle opening and the eye to be treated. It comprises an array of lenses arranged around a dispensing opening which, only at a correct position, allow recognition of a specific image located behind the lenses. If the image is recognizable, the user triggers the pulse of liquid. However, if the user suffers from impaired vision, it may be difficult to recognize the image. Said document also discloses an auxiliary in the form of a concave mirror having a central hole. Said mirror is chosen such that, at the correct distance and angle, the user will see their eye in focus in the mirror. Owing to the magnification of the mirror, a user with impaired eyesight shall also be able to use the auxiliary. However, this additionally requires that the user have a comparatively steady hand in order to also be able to actuate the wetting device when the correct position in relation to the eye has been reached. Moreover, soiling of the auxiliaries owing to wetting by treatment liquid cannot be ruled out.

It is therefore an object of the invention to provide a device and a method that both avoid the disadvantages of the prior art.

The invention shall therefore allow the self-performed dispensing of a precise amount of a liquid comprising a medicament to the eye of a user, specifically even if the user only has reduced vision and/or limited manual dexterity.

The object is achieved by a device according to the first or second aspect of the invention, by a method according to third aspect of the invention as well as a computer program product and computer implemented method according to the fourth and fifth aspect of the invention. Advantageous embodiments can be found in the respectively dependent claims, in the description which follows and in the figures.

### SUMMARY OF THE INVENTION

In a first aspect, the invention relates to a device (1) for wetting an eye (2) with a liquid (F), the device (1) comprising a reservoir (3) for storage of the liquid (F), a dispensing device (4) which is feedable from said reservoir (3) and which is orientable to the eye (2), and a triggering device (5) which activates same, wherein the device (1) comprises or is connectable to a means for sensing (6) of a suitable position of the dispensing device (4) in relation to the eye (2) for wetting.

In a second aspect, the present invention provides for a dispensing unit (8) comprising a reservoir (3), a dispensing device (4) and a triggering device (5) for a device (1) according to the first aspect of the invention.

In a third aspect, the invention provides for a method for wetting an eye (2) with a liquid (F), comprising the following step:
continuously detecting the position of a dispensing device (4) designed to withdraw a liquid (F) from a reservoir (3) and to use it for wetting the eye (2); wherein a signal is transmitted when a suitable position has been sensed using a means for sensing (6) of a suitable position of the dispensing device (4) in relation to the eye for wetting.

In a fourth aspect, the present invention provides for a computer program product which is installable on a device (1) according to the first aspect of the invention and is designed to carry out the following steps when executed as part of a method according to the third aspect of the invention:
(i) processing the image of the eye (2) captured by the means for sensing (6),
(ii) sensing a suitable position of the dispensing device (4) in relation to the eye (2) for wetting, and
(iii) transmitting the signal when a suitable position has been sensed.

In a fifth aspect, the present invention provides for a computer implemented method causing a device (1) according to the first aspect of the invention to carry out the following steps when executed as part of a method according to the third aspect of the invention:
(i) processing the image of an eye (2) of a subject captured by the means for sensing (6),
(ii) sensing a suitable position of the dispensing device (4) in relation to the eye (2) for wetting, and
(iii) transmitting the signal when a suitable position has been sensed.

### DESCRIPTION OF THE DRAWINGS

FIG. 1 shows the side view of a schematic representation of a first embodiment of the device according to the invention;

FIG. 2 shows the side view of a schematic representation of a second embodiment;

FIG. 3 shows the side view of a schematic representation of a third embodiment;

FIG. 4 shows the side view of a schematic representation of a fourth embodiment;

FIGS. 5, 6 show the front view and side view of a schematic representation of one embodiment with marking with the eye in a non-suitable position;

FIGS. 7, 8 show the front view and side view of this further embodiment with the eye in a suitable position;

FIG. 9 shows one embodiment, according to which the marking is passively depicted on the screen by a mirror image of the eye;

FIG. 10 shows one embodiment, according to which the marking is provided by the camera of the means for sensing

FIG. 11 shows a side view of a schematic representation of the third embodiment in which the first separate unit and the second separate unit have corresponding connecting means.

### DETAILED DESCRIPTION OF THE INVENTION

The device according to the invention will be described first, followed by the dispensing device for such a device, the method according to the invention and the associated computer program product or computer implemented method.

Unless the context clearly indicates or requires otherwise, the words ‘comprise’, ‘comprises’ and ‘comprising’ and similar expressions are to be construed in an open and inclusive sense, as ‘including, but not limited to’ in this description and in the claims.

The term ‘a’ or ‘an’ does not exclude a plurality; i.e. the singular forms ‘a’, ‘an’ and ‘the’ should be understood as to include plural referents unless the context clearly indicates or requires otherwise. In other words, all references to singular characteristics or limitations of the present disclosure shall include the corresponding plural characteristic or limitation, and vice versa, unless explicitly specified otherwise or clearly implied to the contrary by the context in which the reference is made. The terms ‘a’, ‘an’ and ‘the’ hence have the same meaning as ‘at least one’ or as ‘one or more’ unless defined otherwise.

The expressions, ‘one embodiment’, ‘an embodiment’, ‘a specific embodiment’ and the like mean that a particular feature, property or characteristic, or a particular group or combination of features, properties or characteristics, as referred to in combination with the respective expression, is present in at least one of the embodiments of the invention. The occurrence of these expressions in various places throughout this description do not necessarily refer to the same embodiment. Moreover, the particular features, properties or characteristics may be combined in any suitable manner in one or more embodiments.

In a first aspect, the present invention provides for a device for wetting an eye with a liquid, the device comprising a reservoir for storage of the liquid, a dispensing device which is feedable from said reservoir and which is orientable to the eye, and a triggering device which activates same, wherein the device comprises or is connectable to a means for sensing of a suitable position of the dispensing device in relation to the eye for wetting.

The device is used for wetting of the eye with a liquid, such as water or an aqueous solution, that contains in particular a medicinal preparation.

The liquid to be dispensed by the device according to the present invention, preferably to a surface of an eye of a subject, may be water, preferably sterilized water or an aqueous or non-aqueous liquid composition comprising water or a pharmaceutically acceptable organic solvent or a mixture of water and one or more organic solvents as an aqueous vehicle. The term “liquid” as used herein is to be understood broadly to include liquids with a broad range of viscosities such as liquids with a very low viscosity in the range of, for example, from about 0.1 cP (centipoise) to about 1 cP as well as liquids with a very high viscosity in the range of, for example, from about 100 to about 200 cP. In some embodiments, the liquid to be dispensed by the device according to the present invention may have a viscosity in the range of from about 1 cP to about 100 cP or to about 75 cP or to about 60 cP (whereas 1 cP corresponds to 0.001 Pa*s (pascal-second)).

The liquid as referred to herein may also comprise further constituents such as pharmaceutically active ingredients and excipients such as salts, preservatives, buffers and the like. In preferred embodiments, the pharmaceutically active ingredients optionally comprised by the liquid to be dispensed by the device according to the present invention are suitable for the treatment of an ophthalmic disease or disorder and for the topical administration to the eye of a subject in need thereof. In further embodiments, the further constituents of the liquid as mentioned above are suitable for administration to the eye of a subject. In yet further embodiments, the liquid to be dispensed by the device according to the present invention is provided in sterilized form. The term “subject” as used herein preferably refers to human or animal, preferably a warm-blooded animal, especially to a human.

The device comprises a reservoir for storage of said liquid. The reservoir can be fixedly connected to the device, but can be preferably exchangeable. It can be under pressure, though it is preferably without pressure, and it comprises for example a collapsing bag.

In some embodiments, the reservoir is provided in the form of a cartridge comprising a dimensionally stable outer casing and an inner container comprising the liquid to be dispensed in the form of a collapsible bag. In further embodiments, the reservoir may be comprised by the dispensing unit as described in further detail below either in the form of a non-exchangeable reservoir or as an exchangeable reservoir or cartridge.

The device further comprises a dispensing device which is feedable from said reservoir and which is orientable to the eye. The dispensing device is therefore used for targeted dispensing of liquid, preferably to the eye or, more specifically to the surface of the eye of a subject. In some embodiments, the dispensing device comprises a nozzle or a nozzle array that the liquid leaves under a certain speed, for example from 0.5 to 10 m/s. This speed may be achieved by means of an accelerating device which accelerates the liquid. The accelerating device can comprise mechanical means such as a tensible spring or a pressurized container with valve, or else electrical means such as a pump or a vibrating mesh, that launches wetting liquid. Orientation is achieved in the simplest case by manual movement of the entire device by the user, but can also be achieved jointly with a few components thereof, or by separate orientation of merely the dispensing device, and here in particular the nozzle or the nozzle array.

In some embodiments, the dispensing device may comprise a mechanical or electromechanical pump, for example, a check-valve pump, a peristaltic pump, a rotary pump, a valveless rectification pump or an ultrasonic pump. In further embodiments, the dispensing device may comprise a non-mechanical pump, for example an electrohydrodynamic pump, an electrokinetic pump, a phase transfer pump or a magnetohydrodynamic pump. However, in further embodiments, the dispensing device may comprise other pump systems like syringe-like pump systems, screw pumps, gear pumps, heat-based pumps or piezo electric pumps.

Moreover, the device comprises a triggering device which activates the dispensing device (and here in particular the accelerating device). The triggering device has the task of triggering the dispensing device (spring, pressurized container valve) or putting it into operation (pump, mesh) at a specific time. The triggering device is not to be confused with a general “activation device” of the device that is typically also present and that is merely used to change said device from an idle mode to an active mode (e.g. on/off switch). Only after the device has been switched on can it actually be used in the manner according to the invention, so as to avoid unintended use.

In some embodiments, the triggering device is adapted to be directly (automatically) actuated by the means for sensing as described in further detail below. In alternative embodiments, however, the triggering device may be adapted to be actuated indirectly, for example, manually by an operation of the subject (to the eye of which the liquid is to be administered) or by another user, for example by operating an actuator provided on the device by providing an acoustic command.

In some embodiments, the device according to the invention may comprise a means for sensing of a suitable position of the dispensing device in relation to the eye for wetting (hereinafter sometimes also referred to as “means for sensing”).

In alternative embodiments, however, the device according to the invention may be connectable to a means for sensing of a suitable position of the dispensing device in relation to the eye for wetting. In other words, the device or, more specifically, the dispensing device according to the present invention, in some embodiments, is adapted for being connected to the means for sensing a suitable position of the dispensing device in relation to the eye for wetting as described in further detail below. The term “connectable to” as used herein in connection with the means for sensing means that the device according to the present invention can be connected to the means for sensing either by a mechanical or by non-mechanical connection or by both, a mechanical connection in order to mechanically attach or mount the means for sensing to the device and by a non-mechanical connection in order to establish a (wireless) connection between the means for sensing and the device in order to allow for the transfer of data and/or energy between the means for sensing and the device according to the present invention as also described in further detail below.

In some embodiments, however, the present invention provides for a device for wetting the eye with a liquid, the device comprising a reservoir for storage of the liquid, a dispensing device which is feedable from said reservoir and which is orientable to the eye, and a triggering device which activates same, wherein the device comprises a means for sensing of a suitable position of the dispensing device in relation to the eye for wetting.

The means for sensing of a suitable position of the dispensing device in relation to the eye for wetting, in some embodiments, is used to sense whether the device, and here in particular the dispensing unit, is in a position in terms of distance and/or direction in which triggering of the dispensing device would lead to the desired wetting of the eye. This sensing preferably is done automatically, i.e. independently of the user.

If, now, the user holds the device in a suitable position in front of the eye to be treated, the means for sensing that detects this carries out an action which directly (automatically) or indirectly (by means of actuation by the user) leads to triggering of the dispensing device and thus to correct treatment of the eye. It is irrelevant how good the remaining vision or manual dexterity of the user is, since the means for sensing operates completely independently thereof. Provided that the means for sensing has a sufficiently rapid response speed, the device can even be used by long-sighted patients or patients with Parkinson's disease.

The invention therefore avoids the disadvantages known from the prior art.

The invention therefore allows the self-performed dispensing of a precise amount of a liquid comprising in particular a medicament to the eye of a user, specifically even if the user only has reduced vision and/or limited manual dexterity. In addition to this, the device according to the invention allows for the simplified and more precise application of a liquid to the eye of a subject even when the device is operated by a person or user other than the subject.

In some embodiments, the device according to the present invention is provided in a form in which the reservoir, the dispensing device and the triggering device is provided by a first separate unit (herein also referred to as “dispensing unit”). In this context, the expression “separate” should be understood as being reversibly attachable and/or detachable to another unit or component of the device. In particular, the first separate unit is configured to be reversibly attachable to, or physically connectable with, a second separate unit of the device by a user, and vice versa. According to these embodiments, the first separate unit of the device comprises at least the reservoir, the dispensing device and the triggering device as described in detail above. In some embodiments, however, the first separate unite may comprise further components such as fluid connections between the reservoir and the (components) of the dispensing unit as well as electrical connections, circuitry and other electronic components necessary to operate to components provided in the dispensing unit, especially the triggering device and the dispensing device.

In further embodiments, the first separate unit does not comprise the means for sensing of a suitable position of the dispensing device in relation to the eye for wetting, or components thereof.

The dispensing unit as the first separate unit may comprise a housing (hereinafter also referred to as “first housing” or “housing of the dispensing unit”). The housing or casing may be in the form of an at least partially hollow and dimensionally stable body comprising or containing the reservoir or a slot into which an exchangeable reservoir may be introduced, the dispensing device and the triggering device and optional further components of the dispensing unit as described above.

In some embodiments, the first separate unit does not comprise an energy storage. In further embodiments, the first separate unit does not comprise a data storage. In further embodiments, the first separate unit does not comprise a long-term energy storage, for example in the form of a rechargeable or non-rechargeable battery. In some embodiments, however, the first separate unit may comprise one or more electronic components for the temporal storage of electrical energy with a low capacitance, for example in the form of capacitors or supercapacitors. In further embodiments, the first separate unit does not comprise a long term or non-transitory data storage. In some embodiments, however, the first separate unit may comprise a transitory data storage as necessary to operate to components provided in the dispensing unit, especially the triggering device and the dispensing device.

In some embodiments of the device of the present invention, the means for sensing of a suitable position of the dispensing device in relation to the eye for wetting is provided by, or as part of, a second separate unit (herein also referred to as “sensing unit”).

According to these embodiments, the second separate unit comprises at least the means for sensing of a suitable position of the dispensing device in relation to the eye for wetting as described in detail further below. In some embodiments, however, the second separate unit may comprise further components as described in further detail below including, but not limited to suitable sensors, means for displaying instructions, electrical connections, circuitry and other electronic components necessary to operate to components provided in the second separate unit.

In further embodiments, the second separate unit does not comprise either one of the reservoir, the triggering device and the dispensing device or components thereof.

The second separate unit may comprise a housing (hereinafter also referred to as “second housing” or “housing of the sensing unit”). This housing or casing may be in the form of an at least partially hollow and dimensionally stable body comprising or containing the components of the second separate unit as also further described below.

In some embodiments, the second separate unit comprises an energy storage, specifically a long-term energy storage, for example in the form of a rechargeable or non-rechargeable battery. In some embodiments, the rechargeable or non-rechargeable battery of the second separate unit provides for the supply of electrical energy to the second separate unit as well as to the first separate unit.

In further embodiments of the device of the present invention, the first separate unit and the second separate unit each comprise connecting means to form a detachable mechanical connection to each other in a connected state. In some embodiments, the connecting means provided in the first separate unit reciprocally fit to the connecting means of the second separate unit and are provided in a form to allow for the generation of a mechanical connection between the first separate unit and the second separate unit. In preferred embodiments, the connecting means provided in the first separate unit reciprocally fit to the connecting means of the second separate unit and are provided in a form to allow for the generation of a detachable mechanical connection between the first separate unit and the second separate unit to allow for the repeated connection and disconnection of the two separate units to/from each other.

In yet further embodiments, when connected to each other (herein also referred to “in the connected state”) the first separate unit and the second separate unit form a mechanically stable assembly. The term “mechanically stable assembly” as used herein means that the first and the second separate unit in the connected state cannot be moved (for example by translation or rotation) independently from each other by a user or subject. This means that if a user or subject holds only one of the first and second separate unit in the connected state a corresponding rotational or translational movement of both separate units will result from a translational or rotational operation by the user or subject. This mechanically stable assembly allows for the facilitated and more precise handling of the device of the present invention especially with regard to the orientation of components of the device, specifically in cases in which components that are comprised by the first separate unit such as the dispensing device and a component comprised by the means for sensing such as the camera or the screen as described in further detail below are to be precisely oriented towards the same object such as the eye of a user or subject.

In further embodiments of the present device, the first separate unit and the second separate unit in the connected state are provided in a defined position relative to each other.

In some embodiments, the connecting means may be in the form of suitable corresponding structures on the outer surface of the first and the second separate unit, for example in the form of preferably detachable form—or force-fitting structures such as plug connectors or guide rails. However, in general, further technical solutions may also be provided like magnetic connectors or adhesive connectors. In specific embodiments, however, the connecting means comprise a plug connection. In this context, a plug connection should be understood as an electromechanical plug connection which is adapted to provide both a mechanical and an electronic data connection.

In yet further embodiments of the present device, the connecting means comprise an interface for the transfer of data and/or energy between the first separate unit and the second separate unit. In particular embodiments, such an interface for the transfer of data between the first separate unit and the second separate unit in both directions may be contactless, for example by a Bluetooth, RFID or other means of wireless data transfer. In further embodiments, also the transfer of energy, especially from the second separate unit to the first separate unit (dispensing unit) may be provided via a contactless connection, for example by a wireless inductive power transfer.

In particular embodiments, however, the interface for the transfer of data and/or energy between the first separate unit and the second separate unit is provided in the form of a mechanical connection, specifically by a plug connection. In some embodiments, plug connectors may be provided that allow for both, the transfer of data and energy in both directions between the first and second separate unit. Suitable plug connectors in this regard comprise but are not limited to, for example, interfaces of the USB-type, especially micro-USB and USB-C as well as Lightning®. Accordingly, in particular embodiments, the connecting means as well as the interface for the transfer of data and/or energy between the comprises USB or Lightning connectors, specifically USB-C connectors. Such corresponding Lightning or USB-C connectors may be provided on the first separate unit as well as on the second separate unit. In this regard, however, it should be noted that the connectors as referred to herein may be replaced by future connectors suitable for the exchange of data and/or energy, accordingly.

In specific embodiments, as will be discussed further below, the first separate unit (dispensing unit) may comprise a male (USB, USB-C or Lightning) connector and the second separate unit may comprise a female (USB, USB-C or Lightning) connector.

In further embodiments, the device according to the present invention further comprises a camera.

According to a preferred embodiment, the means for sensing of a suitable position of the dispensing device in relation to the eye for wetting comprises a camera. The camera provides the means for sensing with information which is evaluated by the means and from which actions are derived by said means.

The term “camera” is to be understood broadly in the present case and means any device which can capture a picture of the situation in front of the camera (which picture may also be severely limited), which picture is further processable electronically. The camera can either operate in the visible and/or in the invisible range of light (CCD camera, infra-red camera) or be based on other physical principles (e.g. LIDAR, ultrasound).

According to one embodiment, the device comprises means for emitting of an acoustic signal (e.g. loudspeaker, beeper) when a suitable position for wetting has been sensed. This means that the user is played the acoustic signal when the device is in a suitable position, whereupon the user actuates the device, more precisely the triggering device, and so the desired amount of liquid is dispensed at the right site, namely the eye to be treated, at the right moment.

According to another embodiment combinable therewith, the device comprises means for emitting of an optical signal (e.g. LED, flash) when a suitable position for wetting has been sensed. This means that the user is shown the optical signal when the device is in a suitable position, whereupon the user actuates the triggering device.

According to another embodiment likewise combinable with the preceding embodiments, the device comprises a screen. It can also be the means for emitting of an optical signal, i.e. the screen illuminates or flashes as a whole, or symbols appropriately indicating that the correct position has been reached are depicted. The screen should be of a format allowing it to be looked at while the dispensing device is in the range of the eye to be treated.

In some embodiments, the screen may be comprised by the means for sensing of a suitable position of the dispensing device in relation to the eye for wetting, or by the second separate unit (i.e. the sensing unit). The term “screen” as used herein preferably refers to a display that allows for the variable graphical representation of optical signals and pictures and accordingly, is not to be confused with a fixed array of single luminous spots, for example LEDs.

In further embodiments of the device of the present invention, the camera and the screen are (both) comprised by the means for sensing of a suitable position of the dispensing device in relation to the eye for wetting, as preferably provided in the second separate unit.

In some embodiments, the screen as well as the camera may be provided on suitable positions or outer surfaces of the device of the present invention, especially on suitable outer surfaces of the sensing unit (i.e. the second separate unit of the device). In particular embodiments, the screen is provided on the same side or outer surface of the sensing unit as the camera.

In further embodiments of the device according to the present invention, the screen and the camera are oriented in the same direction, especially when both are provided on the same side and/or outer surface of the sensing unit as described above. This configuration, in some embodiments, facilitates the orientation of the screen and the camera to an object such as the face or an eye of a subject when using the device of the present invention.

In alternative embodiments, the device comprises a user facing side and a side which is opposite of the user facing side and the screen may be provided on one side or outer surface of the sensing unit and the camera may be provided on another side or surface of the sensing unit, for example on an opposite surface. In these embodiments, the screen and the camera are not oriented in the same direction, but are oriented in different, in some embodiments even in opposite directions. In these embodiments, the device according to the present invention is especially suitable for wetting the eye of a subject in a mode in which the device is not operated by the subject but by a different person, for example by a person assisting the subject in the administration of the liquid to the eye of the subject.

In some embodiments, the device according to the present invention comprises more than one camera, for example two or even three or more cameras as it is often the case in current portable mobile devices, especially mobile telephones as described in further detail below. In these embodiments, the device may comprise one camera which is used for sensing of a suitable position of the dispensing device in relation to the eye for wetting (herein also referred to as “active camera”) along with at least one further camera which is currently not used for sensing of a suitable position of the dispensing device in relation to the eye for wetting. In these embodiments, the device according to present invention may be used in or conveniently be switched between different operating modes. In one operating mode, for example, the relevant or active camera (of the two or more cameras) is oriented towards the same direction as the screen which is an advantageous configuration of the device of the present invention for self-administration of the liquid to the eye of a subject by the subject itself. In another operating mode, the relevant or active camera (of the two or more cameras) is oriented towards a different, specifically towards the opposite direction of the screen which is an advantageous configuration of the device of the present invention for an assisted wetting of an eye of a subject by a person different from the subject.

As outlined above, the dispensing unit which may be provided as the first separate unit may be connected to the second separate unit in a connected state of the device of the present invention and comprises a dispensing device. In some embodiments of the device of the present invention, in the connected state the dispensing device is located on the same side of the device as the means for sensing of a suitable position of the dispensing device in relation to the eye or wetting as the camera. In some embodiments of the device of the present invention, in the connected state the dispensing device is located on the same side of the device as the camera and the screen. In alternative embodiments, however, in the connected state the dispensing device is located on the same side of the device as the camera and on the opposite side of the screen. In further embodiments, the dispensing device is located on the same side of the device and is oriented in the same direction as the active camera. In further embodiments, the dispensing device is located on the side of the device and is oriented in the same direction as the screen and the active camera. In yet further embodiments, a nozzle or a nozzle array that the liquid to be administered to an eye of a subject leaves under a certain speed as comprised by the dispensing unit, more specifically the dispensing device, is oriented or orientable towards the same direction as the camera or active camera of the device according to the present invention. In further embodiments, a nozzle or a nozzle array that the liquid to be administered to an eye of a subject leaves under a certain speed as comprised by the dispensing unit, more specifically the dispensing device, is oriented or orientable towards the same direction as the camera or active camera and the screen of the device according to the present invention.

As described above, the means for sensing of a suitable position of the dispensing device in relation to the eye for wetting, as may be provided by the second separate unit, in some particular embodiments may comprise a camera and/or a screen. In some embodiments, however, the means for sensing of a suitable position of the dispensing device in relation to the eye for wetting comprise one or more further components which may be independently selected from the group comprising a processor, a transitory or non-transitory data storage, an energy storage such a rechargeable or non-rechargeable battery, a microphone, a means for emitting an acoustic signal, means for generating a haptic effect such as a vibration signal as generated by a vibrator, light emitting elements, sensors such sensors for temperature and (air) humidity, and others. In further embodiments, these components may be comprised by the means for sensing in addition to the further potential components as described above such as the screen, the camera, the connecting means, the housing and further mechanical components as necessary.

According to one embodiment, all of the components mentioned, if actually provided, may be housed in a common housing. This preferably also applies to all other components which are required for operation, but not explicitly mentioned (e.g. the energy supply). The advantage of such a device lies in the optimal coordination of all components with one another; in this way, a smallest possible device is also providable.

According to another embodiment as already described above, the reservoir, the dispensing device and the triggering device are arranged in a separate housing. They form a first separate unit which must then be suitably connected, for example wirelessly or non-wirelessly, to the means for sensing of a suitable position for wetting, which means preferably comprises a camera. The advantage of this embodiment is that standardized components can be used for the components not present in the housing of the unit (camera, means for sensing, screen, . . . ) , which can reduce the overall costs. Moreover, in the event of damage to one of the components of the unit, the entire device does not need to be replaced at once.

According to a preferred embodiment, the camera, the means for sensing of a suitable position for wetting, and possibly further components as described above that are not included in the above-defined “first separate unit”, are part of a portable mobile device, or, in other words, the means for sensing of a suitable position of the dispensing device in relation to the eye for wetting is provided by a portable mobile device. The term “portable mobile device” as used herein is to be interpreted broadly as an electronic device that can be operated when held in the hand or hands of a user and can be carried by such user without exceptional efforts. Examples of such portable mobile devices comprise but are not limited to tablet computers, netbooks, laptops, digital media players, handheld personal computers, handheld game consoles, mobile phones, phablets, wearable computers such as smartwatches and others.

The “peripherals components” are then contained in such a device and can be made usable in a suitable manner by suitable software and by use of known interfaces (USB, Bluetooth, RFID, . . . ). The housing of the first separate unit is, in this case, preferably mechanically detachably connectable to the mobile device, for example by means of a shell to which the separate unit is attached and into which the mobile device is insertable or pluggable, or by the separate unit being pluggable onto the mobile device, as already described above.

Particularly preferably, the portable mobile device is a mobile telephone (“smartphone”). Such a device typically comprises all the component parts mentioned above as peripherals components, and a processor of sufficient power for the described task of sensing of a suitable position by means of evaluation of the camera image. Furthermore, as described in detail above, the mobile phone preferably comprises a screen and a camera.

According to one embodiment, the device, when comprising a screen, may be configured to depict in the region of the screen a marking which, upon fixation with the eye of the user, results in a suitable position for wetting. This means that, when said marking has been precisely fixed, the device is at the same time also correctly oriented and distanced in relation to the eye. If the geometric relationship between, firstly, the position and orientation of the dispensing device and, secondly, the marking is known, the latter can be displayed exactly where its fixation leads to said suitable position. A calibration beforehand, which reveals the distance at which a user must hold the screen in order to view it in focus, may be helpful. If the marking is fixed and, as a result, recognized as being in focus, what is especially also observed is the distance from the screen that is relevant to the correct position.

According to one embodiment, the marking is actively depictable on the screen. This means, firstly, that it is not the screen as a whole that is used to emit the optical signal, but only a portion thereof. In particular, it means that the screen is activated such that the marking is depicted on the screen by pixels. The marking can be, for example, a reticule, a dot or a cross. The marking can be depictable at a fixed site of the screen, but can preferably also be movingly or movably/changeably depictable. In this way, the location of the marking can, for example, indicate a direction into which the device is to be moved/tilted by the user in order to arrive at the suitable position. This can be done in a simple manner, for example by means of arrows.

According to another embodiment, the marking is passively depictable on the screen by a mirror image of the eye. The screen, or the mirror image, is therefore used to depict the (main) marking to be fixed. If it is shown exactly in focus, the distance from the screen is known (but may need to be calibrated beforehand). The advantage is that the distance from the screen is halved compared to the preceding embodiment. This leads to a significant reduction in the distance of the device from the eye, which can result in more reliable dispensing of the liquid at the desired site.

Optionally, the device is configured to actively depict on the screen an auxiliary marking which, at a suitable position, correlates with the mirror image of the eye, for example surrounds it or shows a reticule on the pupil. Although said auxiliary marking will possibly not be perceived as being in focus, it will still be readily recognized such that the user is given a sufficiently clear signal as to when the suitable position has been reached, or in what direction the screen is to be moved to reach the suitable position.

According to one embodiment associated with the preceding embodiment, the marking is provided by the camera that is then present. The user therefore looks straight onto the above-mentioned camera with the eye to be treated, which ensures that said camera also has the eye to be treated in the field of view. If the dispensing device is at a short distance from the camera, the path to be bridged by the liquid is also very short, which promotes a short response time.

According to a further embodiment, the device is configured to measure the distance from the eye and/or it comprises position sensors by means of which the orientation of the device is sensible. For example, the camera can comprise an autofocus which is used for distance measurement, or the above-mentioned LIDAR is used for this purpose. The position sensors are used for sensing the orientation of the device in space; this information, too, may be relevant for the correct calculation of the activation of the dispensing device; activation in the case of an upward directed dispensing device thus differs from that of a horizontally oriented dispensing device.

According to a further embodiment, the device is configured to output an optical and/or acoustic signal to guide the user to the suitable position. Such optical signal preferably is generated by/displayed on the screen as preferably comprised by the sensing unit. It can, for example, be provided in the form of an optical “light dot” which is perceived nevertheless by even a user with poor eyesight; when the brightness is maximal, said user knows that the correct position has been reached. An acoustic signal which rises or falls can also be usable here in a supportive manner.

The signal can be continuous or intermittent; by means of tone frequency or tone intervals or light colour/brightness or flashing frequency, it can “encode” the change relative to the suitable position, i.e. indicate whether the user is getting closer to or moving away from said position.

According to one embodiment, the means for sensing is not directly connected to the triggering device, i.e. the user actuates said triggering device manually. Accordingly, in some embodiments, the device of the present invention may be configured for manual actuation of the triggering device when a suitable position of the dispensing device in relation to the eye for wetting has been sensed, for example by pressing a button or speaking a voice command by the subject or user or by another person.

According to a preferred embodiment, the device is configured to automatically actuate the triggering device when a suitable position of the dispensing device in relation to the eye for wetting has been sensed. In other words, the means for sensing of a suitable position of the dispensing device in relation to the eye for wetting is connectable or connected to the triggering device, and activates at the right moment the triggering device of the dispensing device feedable from the reservoir, without the user themself having to actively engage or activate the triggering mechanism. The advantage of this embodiment is that even users with physical limitations, and in particular those with a slowed response time, can still use the device without any problems. In fact, such a user can repeatedly bring the device in an unintentionally different location over and over again near the correct position; when the correct position has then been reached (by chance), automatic triggering lastly occurs without the user having to manually actuate the triggering device.

The invention also relates to a housing in accordance with the above definition of a “separate unit”; to avoid repetition, reference is made to the above passages.

Accordingly, in a second aspect the present invention provides for a dispensing unit comprising a reservoir, a dispensing device and a triggering device for a device according to the first aspect of the invention. In some embodiments of this aspect, the present invention relates to a dispensing unit comprising a reservoir, a dispensing device and a triggering device for a device according to the first aspect of the invention which is adapted to be coupled or which is suitable to be coupled or which is configured to be coupled to a means for sensing of a suitable position of the dispensing device in relation to the eye for wetting, especially in cases in which such means for sensing of a suitable position of the dispensing device in relation to the eye for wetting is provided in the form a portable mobile device as described in connection with the first aspect of the invention, specifically in the form of a portable mobile device such as a handheld portable mobile device, for example a mobile telephone or tablet computer. In some embodiments, the dispensing unit of this aspect of the invention corresponds to the first separate unit of the device of the first aspect of the invention which preferably is provided in a first separate housing as described in connection with the first aspect of the invention. For the avoidance of doubt, any features, embodiments or combinations thereof related to the dispensing unit of the first aspect of the invention apply to this aspect of the invention, accordingly.

The dispensing unit according to this aspect of the invention allows for maximum flexibility with regard to the form and technical nature in which the means for means for sensing of a suitable position of the dispensing device in relation to the eye for wetting is provided. In embodiment, in which the means for sensing is provided in the form of a portable mobile device as described in detail above, the dispensing unit may be provided in a form which allows for the connection with a broad variety of different portable mobile devices.

Furthermore, the dispensing device according to this aspect of the invention allows for the implementation of advantageous forms of the device of the present invention in which the dispensing unit can be provided as a disposable unit which can either contain replaceable reservoir or which can be discarded together with a partially or completely emptied reservoir while all components of the device as comprised by the means for sensing (as preferably comprised by the second separate unit preferably in the form of a portable mobile device) are available for repeated use.

In a third aspect, the invention also relates to a method for wetting the eye with a liquid such as water or an aqueous solution that contains in particular a medicinal preparation.

The method comprises at least the step of continuously detecting the position of a dispensing device designed to withdraw a liquid from a reservoir and to use it for wetting the eye.

Accordingly, in this aspect the invention provides for a method for wetting an eye of a subject with a liquid, wherein the method is preferably carried out using the device according to the first aspect of the invention, the method comprising the following step:
continuously detecting the position of a dispensing device designed to withdraw a liquid from a reservoir and to use it for wetting the eye;
   wherein a signal is transmitted when a suitable position has been sensed using a means for sensing of a suitable position of the dispensing device in relation to the eye for wetting.

In addition, the present aspect of the invention provides for a corresponding method for detecting a suitable position of a dispensing device for wetting an eye of a subject with a liquid, wherein the method is preferably carried out using the device according to the first aspect of the invention, the method comprising the following step:
continuously detecting the position of a dispensing device designed to withdraw a liquid from a reservoir and to use it for identification of a suitable position for wetting the eye;
   wherein a signal is transmitted when a suitable position has been sensed using a means for sensing of a suitable position of the dispensing device in relation to the eye for wetting.

It is clear that this also requires orientation of the dispensing device in such a way that said dispensing device is also in a suitable position in relation to the eye for wetting; in the simplest case, this orientation is carried out by the user, who moves the dispensing device back and forth in front of the eye. Stationary installation of the dispensing device and movement of the head are equally possible. A further possibility is automatic movement of the dispensing device, and so the liquid can be dispensed in the right direction.

Thereafter, a triggering device which activates the dispensing device is (manually or automatically) triggered.

In other words, the method for wetting an eye according to this aspect of the invention comprises wetting the eye under prior orientation of a dispensing device which dispenses the wetting liquid.

The methods of this aspect of the invention comprise the step that a “signal” is transmitted when a suitable position for wetting has been sensed, specifically using a means for sensing of a suitable position of the dispensing device in relation to the eye. The means for sensing therefore has the property of analysing the current position between dispensing device and eye and, in doing so, of also sensing when the dispensing device is in a suitable position for targeted dispensing, i.e. at an eye-wetting position.

According to the invention, a “signal” is emitted when said suitable position has been sensed. The term “signal” is to be understood broadly in the present case and can mean a signal perceivable by a user and/or further processable electronically.

According to a preferred embodiment, the methods comprise the step of recording an “image” by means of a “camera” which is part of the means for sensing. The “image” can be of an optical nature, but also of an acoustic or other nature, and is used to represent the situation of the position of the eye relative to the dispensing unit, the situation being relevant to further evaluation.

According to one embodiment, the signal is an acoustic and/or an optical signal. Such a signal is receivable by the user in a simple manner and can provide said user with information with respect to a suitable position in relation to the eye for wetting having been achieved.

According to another embodiment which is, however, combinable with the preceding embodiment, the signal is of an electronic nature, meaning that it can be further processed in a simple manner.

According to a further embodiment, a marking which, upon fixation with the eye of the user, results in a suitable position for wetting is depicted on a screen. The marking can, for example, be depicted in the form of a reticule or circle. The marking is suitably positioned on the screen in order to fulfil the above purpose. This may require first carrying out a calibration step once only, comprising testing different positions with correspondingly different locations of the marking and selecting that location at which the operation result corresponds to what is desired.

According to a further embodiment, indicators of a graphical nature that guide the user in the direction of the suitable position are depicted on the screen. The indicators can, for example, be in the form of arrows, or of a marking depicted as displaced, and so the user changes the position highly intuitively until the indicators indicate a suitable position (or, for example, disappear).

According to one embodiment, the signal generated at the moment the suitable position for wetting has been reached is passed on to the triggering device (which is then connected to the means for sensing), which triggering device on its part triggers the dispensing device. This means that the method automatically detects the occupation of a suitable position with the aid of the means for sensing, and then promptly (since it is likely there will be further movement by the user) passes on the signal to the triggering device, which may lead to dispensing of the liquid. If these steps occur sufficiently quickly, for example within less than 0.5 seconds, 0.3 seconds or even 0.1 seconds, it can be assumed that the position, even with slight movements by the user, can continue to be regarded as suitable for allowing the desired wetting.

According to another embodiment, the signal generated at the moment the suitable position for wetting has been reached indicates to the user to manually actuate the triggering device themself. Therefore, automatic triggering does not occur here; instead, what occurs is merely the above-mentioned optical and/or acoustic signalling to the user, who then may actuate the triggering device and thus triggers the wetting. In this context, it should be noted that the term “user” as used in connection with this embodiment may or may not be identical to the subject of which an eye is to be wetted, but may also refer to another subject supporting the method of wetting an eye or for detecting a suitable position for wetting an eye according to the present invention.

Particularly preferably, the method according to this aspect of the invention is carried out using a device as described above in connection with the first aspect of the invention. To avoid repetition, reference is made to the relevant passages. Furthermore, it should be noted that all features, embodiments or combinations thereof as disclosed in connection with the device of the first aspect or the dispensing unit of the second aspect of the invention may apply to the methods of this third aspect, accordingly.

In a fourth aspect, the invention also relates to a computer program product which is installable on a device, preferably on a device according to the first aspect of the invention as described above, preferably on the means for sensing of a suitable position of the dispensing device in relation to the eye for wetting, especially on the second separate unit (i.e. the sensing unit), and which carries out specific steps when executed as part of a method according to the third aspect of the invention as described above.

What are carried out are the steps of (i) processing the image of the eye captured by the means for sensing, (ii) sensing a suitable position of the dispensing device in relation to the eye for wetting, and (iii) transmitting the signal when a suitable position has been sensed.

Accordingly, the present invention also provides for a computer program product which is installable on a device according to the first aspect of the invention and is designed to carry out the following steps when executed as part of a method according to the third aspect of the invention:
(i) processing the image of an eye of a subject captured by the means for sensing,
(ii) sensing a suitable position of the dispensing device in relation to the eye for wetting, and
(iii) transmitting the signal when a suitable position has been sensed.

Accordingly, in specific embodiments, the computer program product is installable on the means for sensing of a suitable position of the dispensing device in relation to the eye (2) for wetting.

Furthermore, according to a fifth aspect, the present invention also provides for a computer implemented method, causing a device according to the first aspect of the invention to carry out the following steps when executed as part of a method according to the third aspect of the invention:
(i) processing the image of an eye of a subject captured by the means for sensing,
(ii) sensing a suitable position of the dispensing device in relation to the eye for wetting, and
(iii) transmitting the signal when a suitable position has been sensed.

Optionally, the computer program product or the computer implemented method is further designed to carry out the steps of (iv) displaying a marking on the screen, and/or (v) automatically activating the triggering device.

It is clear that the computer program product and computer implemented method must be able to suitably activate the above-mentioned components or process the information provided thereby. Particularly preferably, the computer program product and computer implemented method is executable on a processor of a mobile device such as a smartphone.

According to a further embodiment, the computer program product and computer implemented method are designed to identify the person using the device, to retrieve data saved in a data memory with respect to the treatment of said person, and to adjust the nature and/or number of signals for activation. In other words, besides the precise positioning of the dispensing device in front of the eye, it is possible with sufficient quality and resolution accuracy of the means for sensing (camera) for an installed program to be used for detecting whether the intended placement of the liquid is actually suitable for the current user. Thus, suitability can be based on the nature, amount and time of triggering. The program and computer implemented method can thus also increase the safety of use by monitoring or controlling the setting of the correct dose or, if multiple reservoirs are present, of the nature of the agent, right up to blocking of unauthorized users.

The following is a list of numbered embodiments E1.1 to E1.26 comprised by the present invention:

E1.1 Device (1) for wetting the eye (2) with a liquid (F), the device (1) comprising a reservoir (3) for storage of the liquid (F), a dispensing device (4) which is feedable from said reservoir (3) and which is orientable to the eye (2), and a triggering device (5) which activates same, characterized in that the device (1) comprises a means for sensing (6) of a suitable position of the dispensing device (4) in relation to the eye (2) for wetting.

E1.2 Device (1) according to embodiment E1.1, wherein the means for sensing (6) of a suitable position of the dispensing device (4) in relation to the eye (2) for wetting comprises a camera.

E1.3 Device (1) according to embodiment E1.1 or E1.2, comprising means for emitting of an acoustic signal when a suitable position for wetting has been sensed.

E1.4 Device (1) according to embodiment E1.1 or E1.2, comprising means for emitting of an optical signal when a suitable position for wetting has been sensed.

E1.5 Device (1) according to any of the preceding embodiments, further comprising a screen (7).

E1.6 Device (1) according to embodiment E1.5, wherein the device (1) is configured to depict in the region of the screen (7) a marking which, upon fixation with the eye (2) of the user, results in a suitable position for wetting.

E1.7 Device (1) according to embodiment E1.6, wherein the marking is actively depictable on the screen (7).

E1.8 Device (1) according to embodiment E1.6, wherein the marking is passively depictable on the screen (7) by a mirror image of the eye (2), and the device (1) is configured to actively depict on the screen (7) an auxiliary marking which, at a suitable position, correlates with the mirror image of the eye (2).

E1.9 Device (1) according to embodiment E1.2 and embodiment E1.6, wherein the marking (10) is provided by the camera.

E1.10 Device (1) according to any of the preceding embodiments, further configured to measure the distance from the eye (2) and/or comprising position sensors by means of which the orientation of the device (1) is sensible.

E1.11 Device (1) according to any of the preceding embodiments, configured to output an optical and/or acoustic signal to guide the user to the suitable position.

E1.12 Device (1) according to any of the preceding embodiments, configured to automatically actuate the triggering device (5) when a suitable position of the dispensing device (4) in relation to the eye (2) for wetting has been sensed.

E1.13 Device (1) according to any of embodiments E1.1 to E1.12, wherein all of the components are housed in a common housing.

E1.14 Device (1) according to any of embodiments E1.1 to E1.12, wherein the reservoir (3), the dispensing device (4) and the triggering device (5) are present as a separate unit (8).

E1.15. Device (1) according to embodiment E1.14, wherein the camera, the means for sensing (6) of a suitable position for wetting, and possibly further components according to Claims 2 to 12, are part of a portable mobile device.

E1.16 Device (1) according to embodiment E1.15, wherein the portable mobile device is a mobile telephone.

E1.17 Housing (8) containing components according to embodiment E1.14 for a device (1) according to embodiment E1.15 or E1.16.

E1.18 Method for wetting the eye (2) with a liquid (F), comprising the following step:
continuously detecting the position of a dispensing device (4) designed to withdraw a liquid (F) from a reservoir (3) and to use it for wetting the eye (2);
   characterized in that a signal is transmitted when a suitable position has been sensed using a means for sensing (6) of a suitable position of the dispensing device (4) in relation to the eye for wetting.

E1.19 Method according to embodiment E1.18, wherein the signal is an acoustic and/or an optical signal.

E1.20 Method according to embodiment E1.18 or E1.19, wherein a marking which, upon fixation with the eye (2) of the user, results in a suitable position for wetting is depicted on a screen (7).

E1.21 Method according to embodiment E1.20, wherein indicators of a graphical nature that guide the user in the direction of the suitable position are depicted on the screen (7).

E1.22 Method according to any of embodiments E1.18 to E1.21, wherein the signal generated at the moment the suitable position for wetting has been reached is passed on to the triggering device (5), which on its part triggers the dispensing device (4).

E1.23 Method according to any of embodiments E1.18 to E1.21, wherein the signal generated at the moment the suitable position for wetting has been reached indicates to the user to manually actuate the triggering device (5).

E1.24 Computer program product which is installable on a device (1) according to any of embodiments E1.1 to E1.16 and is designed to carry out the following steps when executed as part of a method according to any of embodiments E1.18 to E1.23:
(i) processing the image of the eye (2) captured by the means for sensing (6),
(ii) sensing a suitable position of the dispensing device (4) in relation to the eye (2) for wetting, and
(iii) transmitting the signal when a suitable position has been sensed.

E1.25 Computer program product according to embodiment E1.24, further designed to carry out the steps of:
(iv) displaying a marking on the screen (7), and/or
(v) automatically activating the triggering device (5).

E1.26 Computer program product according to embodiment E1.24 or E1.25, further designed to identify the person using the device, to retrieve data saved in a data memory with respect to the treatment of said person, and to adjust the nature and/or number of signals for activation.

The following is a further list of numbered embodiments E2.1 to E2.47 comprised by the present invention:

E2.1 Device (1) for wetting the eye (2) with a liquid (F), the device (1) comprising a reservoir (3) for storage of the liquid (F), a dispensing device (4) which is feedable from said reservoir (3) and which is orientable to the eye (2), and a triggering device (5) which activates same, wherein the device (1) comprises or is connectable to a means for sensing (6) of a suitable position of the dispensing device (4) in relation to the eye (2) for wetting.

E2.2 Device (1) according to embodiment E2.1, wherein the device (1) comprises a means for sensing (6) of a suitable position of the dispensing device (4) in relation to the eye (2) for wetting.

E2.3 Device (1) according to embodiment E2.1 or E2.2, wherein the reservoir (3), the dispensing device (4) and the triggering device (5) is provided by a first separate unit (8), optionally comprising a first housing.

E2.4 Device (1) according to any one of the preceding embodiments, wherein the means for sensing (6) of a suitable position of the dispensing device (4) in relation to the eye (2) for wetting is provided by a second separate unit (8a), optionally comprising a second housing.

E2.5 Device (1) according to embodiment E2.4, wherein the first separate unit (8) and the second separate unit (8a) each comprise connecting means (11, 11a) to form a detachable mechanical or electromechanical connection to each other in a connected state.

E2.6 Device (1) according to embodiment E2.4 or E2.5, wherein the first separate unit (8) and the second separate unit (8a) in the connected state form a mechanically stable assembly.

E2.7 Device (1) according to embodiment E2.5 or E2.6, wherein in the connected state the first separate unit (8) and the second separate unit (8a) unit are provided in a defined position relative to each other.

E2.8 Device (1) according to any one of embodiments E2.5 to E2.7, wherein the connecting means (11, 11a) comprise an interface for the transfer of data and/or energy between the first separate unit (8) and the second separate unit (8a).

E2.9 Device (1) according to embodiment E2.8, wherein the interface for the transfer of data and/or energy between the first separate unit (8) and the second separate unit (8a) is provided in the form of a mechanical or electromechanical connection.

E2.10 Device (1) according to embodiments E2.5 to E2.9, wherein the connecting means (11, 11a) comprise a plug connection.

E2.11 Device according to embodiment E2.10, wherein the first separate unit (8) comprises a male connector and the second separate unit (8a) comprises a female connector.

E2.12 Device (1) according to any one of the preceding embodiments, further comprising a screen (7).

E2.13 Device (1) according to embodiment E2.12, further comprising a camera.

E2.14 Device (1) according to any one of the preceding embodiments, wherein the means for sensing (6) of a suitable position of the dispensing device (4) in relation to the eye (2) for wetting or the sensing unit (8a) comprises a camera.

E2.15 Device (1) according to any one of the preceding embodiments, wherein the means for sensing (6) of a suitable position of the dispensing device (4) in relation to the eye (2) for wetting or the sensing unit (8a) comprises a screen.

E2.16 Device (1) according to embodiment E2.15, wherein the camera and the screen (7) are comprised by the second separate unit (sensing unit, 8a).

E2.17 Device according to embodiment E2.13 or E2.14, wherein the screen (7) is provided on the same side of the device as the camera.

E2.18 Device according to embodiment E2.17, wherein the screen (7) and the camera are oriented in the same direction.

E2.19 Device according to embodiment E2.17, wherein the screen (7) and the camera are oriented in opposite directions.

E2.20 Device according to any one of embodiments E2.13 to E2.19, wherein in the connected state the dispensing device (4) is located on the same side of the device and is oriented in the same direction as the camera.

E2.21 Device according to any one of embodiments E2.13 to E2.20, wherein in the connected state the dispensing device (4) is located on the same side of the device and is oriented in the same direction as the screen (7) and the camera.

E2.22 Device (1) according to any one of the preceding embodiments, wherein the means for sensing (6) of a suitable position of the dispensing device (4) in relation to the eye (2) for wetting comprise one or more further components selected from the group comprising a processor, a transitory or non-transitory data storage, an energy storage such a rechargeable or non-rechargeable battery, a microphone, a means for emitting an acoustic signal, means for generating a haptic effect such as a vibration signal as generated by a vibrator, light emitting elements and sensors such sensors for temperature and (air) humidity.

E2.23 Device (1) according to any one of the preceding embodiments, wherein the means for sensing (6) of a suitable position of the dispensing device (4) in relation to the eye (2) for wetting is provided by a portable mobile device.

E2.24 Device (1) according to embodiment E2.23, wherein the camera, the means for sensing (6) of a suitable position for wetting, and possibly further components according to embodiments 2 to 13, are part of a portable mobile device.

E2.25 Device (1) according to embodiment E2.23 or E2.24, wherein the portable mobile device is selected from the group consisting of tablet computers, netbooks, laptops, digital media players, handheld personal computers, handheld game consoles, mobile phones, phablets, smartwatches and others.

E2.26 Device (1) according to any one of embodiments E2.23 to E2.25, wherein the portable mobile device is a mobile telephone.

E2.27 Device (1) according to any one of embodiments E2.3 to E2.26, wherein the first separate unit (8) does not comprise at least one of an energy storage, specifically a long-term energy storage, and a data storage, specifically a non-transitory data storage.

E2.28 Device (1) according to any one of the preceding embodiments, comprising means for emitting of an acoustic signal when a suitable position for wetting has been sensed.

E2.29 Device (1) according to any one of the preceding embodiments, comprising means for emitting of an optical signal when a suitable position for wetting has been sensed.

E2.30 Device (1) according to any one of embodiments E2.12 to E2.29, wherein the device (1) is configured to depict in the region of the screen (7) a marking which, upon fixation with the eye (2) of the user, results in a suitable position for wetting.

E2.31 Device (1) according to embodiment E2.30, wherein the marking is actively depictable on the screen (7).

E2.32 Device (1) according to embodiment E2.31, wherein the marking is passively depictable on the screen (7) by a mirror image of the eye (2), and the device (1) is configured to actively depict on the screen (7) an auxiliary marking which, at a suitable position, correlates with the mirror image of the eye (2).

E2.33 Device (1) according to embodiments E2.13 and E2.30, wherein the marking (10) is provided by the camera.

E2.34 Device (1) according to any one of the preceding embodiments, further configured to measure the distance from the eye (2) and/or comprising position sensors by means of which the orientation of the device (1) is sensible.

E2.35 Device (1) according to any one of the preceding embodiments, configured to output an optical and/or acoustic signal to guide the user to the suitable position.

E2.36 Device (1) according to any of the preceding embodiments, configured to automatically actuate the triggering device (5) when a suitable position of the dispensing device (4) in relation to the eye (2) for wetting has been sensed.

E2.37 Device (1) according to any of the preceding embodiments, configured for manual actuation of the triggering device (5) when a suitable position of the dispensing device (4) in relation to the eye (2) for wetting has been sensed.

E2.38 Dispensing unit (8) comprising a reservoir (3), a dispensing device (4) and a triggering device (5) (adapted or configured to be coupled to a means for sensing (6) of a suitable position of the dispensing device (4) in relation to the eye (2) for wetting) for a device (1) according to any one of the preceding embodiments.

E2.39 Method for detecting a suitable position of as dispensing device for wetting an eye of a subject with a liquid (F), wherein the method is carried out using the device according to any one of embodiments E2.1 to E2.37, the method comprising the following step:
continuously detecting the position of a dispensing device (4) designed to withdraw a liquid (F) from a reservoir (3) and to use it for identification of a suitable position for wetting the eye (2);
   wherein a signal is transmitted when a suitable position has been sensed using a means for sensing (6) of a suitable position of the dispensing device (4) in relation to the eye for wetting.

E2.40 Method for wetting an eye (2) of a subject with a liquid (F), wherein the method is carried out using the device according to any one of embodiments E2.1 to E2.37 the method comprising the following step:
continuously detecting the position of a dispensing device (4) designed to withdraw a liquid (F) from a reservoir (3) and to use it for wetting the eye (2);
   wherein a signal is transmitted when a suitable position has been sensed using a means for sensing (6) of a suitable position of the dispensing device (4) in relation to the eye for wetting.

E2.41 Method according to embodiment E2.39 or E2.40, wherein the signal is an acoustic and/or an optical signal.

E2.42 Method according to any one of embodiments E2.39 to E2.41, wherein a marking which, upon fixation with the eye (2) of the user, results in a suitable position for wetting is depicted on the screen (7).

E2.43 Method according to embodiment E2.41, wherein indicators of a graphical nature that guide the user in the direction of the suitable position are depicted on the screen (7).

E2.44 Method according to any one of embodiments E2.39 to E2.43, wherein the signal generated at the moment the suitable position for wetting has been reached is passed on to the triggering device (5), which on its part triggers the dispensing device (4).

E2.45 Method according to any one of embodiments E2.39 to E2.44, wherein the signal generated at the moment the suitable position for wetting has been reached indicates to the user to manually actuate the triggering device (5).

E2.46 Computer program product which is installable on a device (1) according to any of embodiments E2.1 to E2.37 and is designed to carry out the following steps when executed as part of a method according to any of embodiments 2.39 to 2.45:
(i) processing the image of an eye (2) of a subject captured by the means for sensing (6),
(ii) sensing a suitable position of the dispensing device (4) in relation to the eye (2) for wetting, and
(iii) transmitting the signal when a suitable position has been sensed.

E2.47 Computer program product according to embodiment E2.46, wherein the computer program product is installable on the means for sensing (6) of a suitable position of the dispensing device (4) in relation to the eye (2) for wetting.

E2.48 Computer implemented method, causing a device (1) according to any of embodiments E2.1 to E2.37 to carry out the following steps when executed as part of a method according to any of embodiments E2.39 to E2.45:
(i) processing the image of an eye (2) of a subject captured by the means for sensing (6),
(ii) sensing a suitable position of the dispensing device (4) in relation to the eye (2) for wetting, and
(iii) transmitting the signal when a suitable position has been sensed.

E2.49 Computer program product according to embodiment E2.46 or E2.47 or computer implemented method according to embodiment E2.46, further designed to carry out the steps of:
(iv) displaying a marking on the screen (7), and/or
(v) automatically activating the triggering device (5).

E2.50 Computer program product or computer implemented method according to any one of embodiments E2.45 to E2.49, further designed to identify the person using the device, to retrieve data saved in a data memory with respect to the treatment of said person, and to adjust the nature and/or number of signals for activation.

### DETAILED DESCRIPTION OF THE DRAWINGS

In what follows, the invention will be exemplified on the basis of the figures as summarized above.

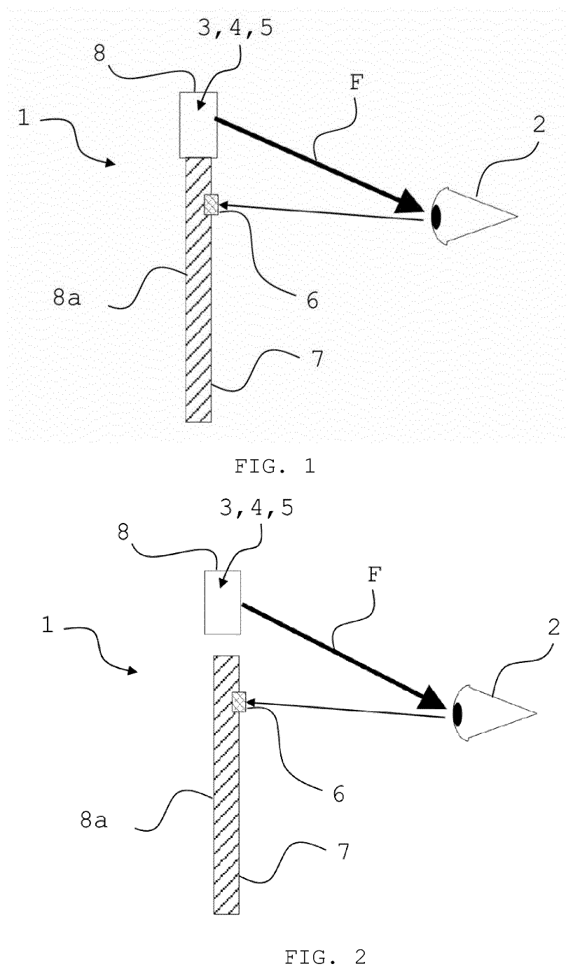

FIG. 1 shows the side view of a schematic representation of a first embodiment of the device according to the invention. The device 1 comprises a reservoir 3 for storage of the liquid F, a dispensing device 4 which is feedable from said reservoir 3 and which is orientable to the eye 2 of a subject, and a triggering device 5 which activates the dispensing device 4 (all three components 3, 4, 5 are depicted combined as a first separate unit or, in other words, dispensing unit 8 in a common housing). FIG. 1 also shows the means for sensing (6) of a suitable position of the dispensing device (4) in relation to the eye (2) for wetting in the form of a second separate unit (8a) comprising a screen (7).

According to the invention, the device 1 comprises a means for sensing 6 of a suitable position of the dispensing device 4 in relation to the eye 2 for wetting, which means is connected to the triggering device 5.

If the means for sensing 6 senses that the eye 2 of the user is in a position in which the eye can be reached by the jet of the liquid F, which is accelerated by a accelerating device not depicted and oriented by a nozzle not depicted, then either a signal (optical or acoustic) indicating manual triggering to the user can be output, or the means for sensing 6 of a suitable position of the dispensing device 3 in relation to the eye 2 for wetting activates at the right moment the triggering device 5 of the dispensing device 4 fed from the reservoir 3, without the user themself having to actively engage or activate the triggering mechanism.

The means for sensing 6 comprises, for example, a camera, indicated by the protruding surface.

The device preferably also comprises a screen 7 which is arranged on the side of the device 1 that faces the user during use of the device 1.

FIG. 2 shows the side view of a schematic representation of a second embodiment. It differs from the embodiment of FIG. 1 in that the housing of the first separate unit 8 is separated from the part of the device 1 that comprises the screen 7 in the form of second separate unit 8a. For use, the first separate unit 8 is preferably mechanically couplable to the second separate unit 8a comprising screen 7.

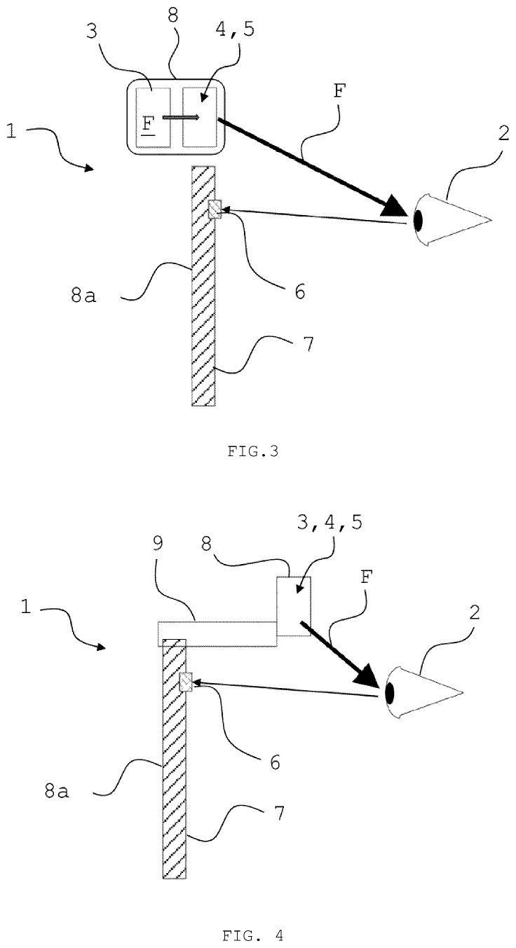

FIG. 3 shows the side view of a schematic representation of a third embodiment, according to which reservoir 3 is separated from the dispensing and triggering device 4, 5; the reservoir is therefore explicitly exchangeable. All of these components remain combined in a common housing of the dispensing unit as a first separate unit 8.

FIG. 4 depicts the side view of a schematic representation of a fourth embodiment. Here, the first separate unit 8 is brought closer to the eye 2 of the user by means of a spacer 9, and so the distance between dispensing device 4 and eye 2 is shorter, which improves the accuracy of the dispensing device 4.

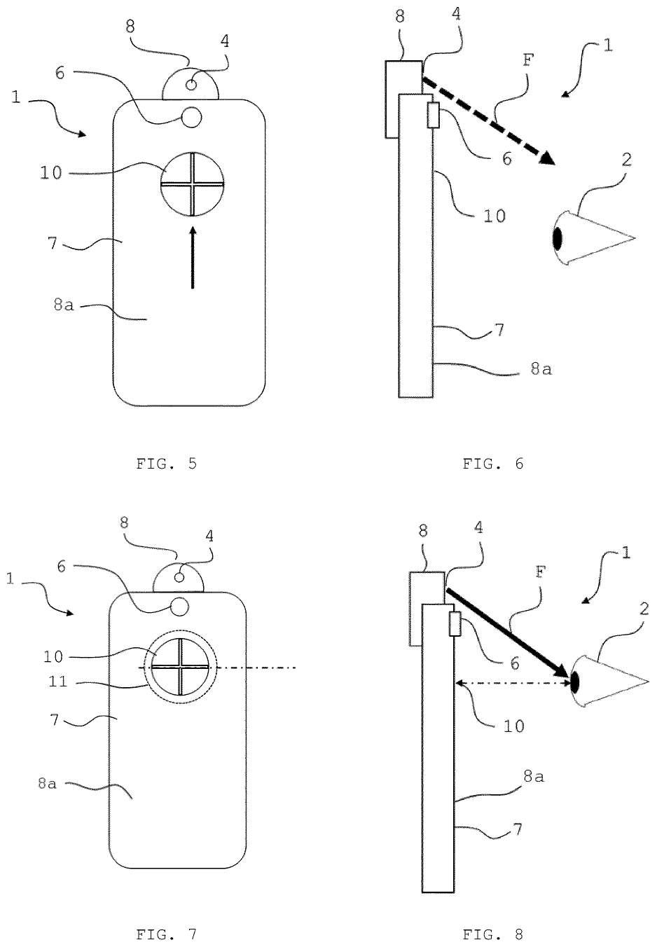

FIGS. 5 and 6 show the front view and side view of a schematic representation of one embodiment with marking 10 on the screen 7 with the eye 2 in a non-suitable position for wetting. In this embodiment, the first separate unit 8 comprising dispensing device 4 is connected to the means for sensing 6 in the form of second separate unit 8a comprising screen 7. FIG. 5 depicts the plan view of the screen 7 of the device 1. It is suitable for depicting a marking 10.

Here, the marking 10 is actively depicted on the screen 7. Further depicted on the screen 7 is an upward directed arrow as guidance indicator (no reference sign). The means for sensing 6 detects the location of the eye 2 relative to the device 1 or the dispensing device 4 and senses that this position is not suitable for dispensing of the liquid F. If, nevertheless, the liquid F were to be dispensed by the dispensing device 4, it would not hit the eye 2; therefore, the corresponding arrow is depicted only in dashed form (side view, FIG. 6).

In the situation shown in FIGS. 7 and 8 by contrast, the eye 2 is in a suitable position, since it has migrated further upwards relative to the dispensing device 4. Now, it is at the same height as the marking 10 (dot-and-dash lines). The marking 10 is positioned such that fixation thereof with the eye 2 of the user results in a suitable position for wetting. This, in turn, is detected by the means for sensing 6, which then triggers the dispensing device 4 by means of the triggering device 5 (not shown), and so the eye 2 is wetted with liquid F (solid arrow).

In parallel, the screen of the embodiment shown additionally displays an auxiliary marking 11 (dashed circle around the marking 10) which shows the user that the device 1 is now in a suitable position. Consequently, instead of using the automatic triggering, it would also be possible for the user to manually actuate the triggering device (alternative embodiment), for example by pressing a button, speaking a voice command, or relatively long closure and subsequent opening of the eye to be treated; such gestures can be detected in a simple manner by means of a camera which can preferably be comprised by the means for sensing 6.

FIG. 9 depicts one embodiment, according to which the marking 10 is passively depicted on the screen 7 by a mirror image 2′ of the eye 2. If it is shown exactly in focus, the distance from the screen 7 is known. The user can therefore hold the device away from their eye 2 just far enough until the mirror image 2′ appears in focus. They are then distinctly closer to the screen 7 compared to the embodiment according to FIGS. 5 to 9. Whereas the length of the dot-and-dash lines in FIG. 8 and FIG. 9 is identical (distance of eye 2 from marking 10 in FIG. 8, distance between eye 2 and mirror image 2′ in FIG. 9), the thick arrow indicating the liquid jet is now distinctly shorter in FIG. 9, which is advantageous for targeted dispensing of the liquid F.

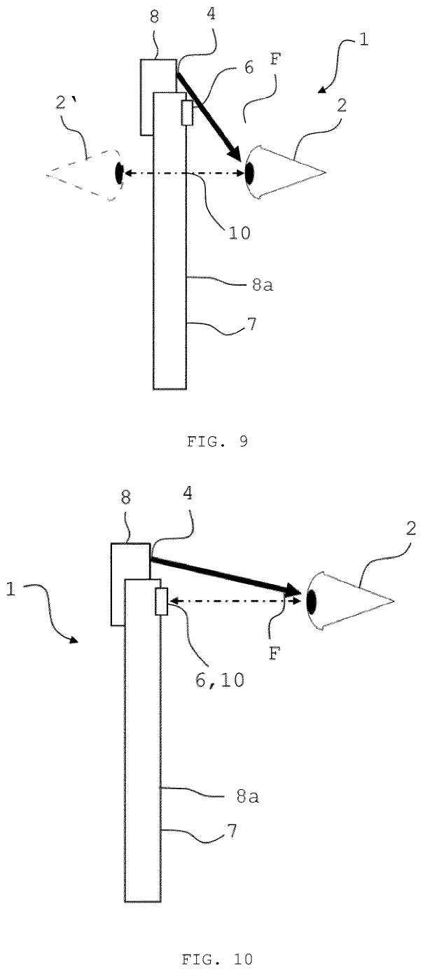

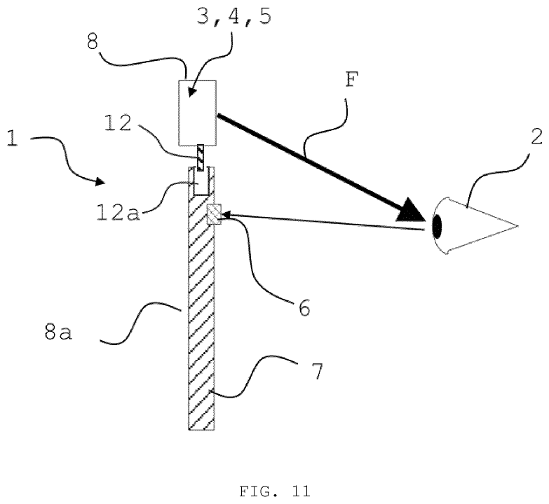

According to the embodiment shown in FIG. 10, the marking 10 is provided by the camera of the means for sensing 6. The user therefore looks straight onto the camera with the eye 2 to be treated, it being advantageous, but not absolutely necessary, if said user also actually sees said camera in focus; it must be at least roughly recognizable (the means for sensing 6 automatically detects the occupation of the suitable position for dispensing). The advantage of this embodiment is that it is ensured that the camera also actually has the eye to be treated in the field of view.

FIG. 11 shows a side view of an embodiment of the device as shown in FIG. 2 in which the first separate unit 8 (dispensing unit) and the second separate unit 8a have corresponding connecting means 12, 12a to establish a mechanically stable connection between the separate units in a connected state. Specifically connecting means 12 and 12a are provided in the form of plug connectors, wherein the connecting means 12 of the first separate unit 8 is provided in the form of a male connector and connecting means 12a of the second separate unit 8a is provided in the form of a female connector.

### LIST OF REFERENCE SIGNS

1 Device
2 Eye
2′ Mirror image
3 Reservoir
4 Dispensing device
5 Triggering device
6 Means for sensing
7 Screen
8 First Separate unit; dispensing unit
8a Second Separate unit
9 Spacer
10 Marking
11 Auxiliary marking
12 Connecting means of the first separate unit
12a Connecting means of the second separate unit
F Liquid

1. Device (1) for wetting an eye (2) with a liquid (F), the device (1) comprising a reservoir (3) for storage of the liquid (F), a dispensing device (4) which is feedable from said reservoir (3) and which is orientable to the eye (2), and a triggering device (5) which activates same, wherein the device (1) comprises or is connectable to a means for sensing (6) of a suitable position of the dispensing device (4) in relation to the eye (2) for wetting.

2. Device (1) according to claim 1, wherein the device (1) comprises a means for sensing (6) of a suitable position of the dispensing device (4) in relation to the eye (2) for wetting.

3. Device (1) according to claim 1 or 2, wherein the reservoir (3), the dispensing device (4) and the triggering device (5) are provided by a first separate unit (8).

4. Device (1) according to any one of the preceding claims, wherein the means for sensing (6) of a suitable position of the dispensing device (4) in relation to the eye (2) for wetting is provided by a second separate unit (8a).

5. Device (1) according to claim 3 and 4, wherein the first separate unit (8) and the second separate unit (8a) each comprise connecting means (11, 11a) to form a detachable mechanical connection to each other in a connected state.

6. Device (1) according to claim 5, wherein the first separate unit (8) and the second separate unit (8a) in the connected state form a mechanically stable assembly.

7. Device (1) according to claim 5 or 6, wherein the connecting means (11, 11a) comprise an interface for the transfer of data and/or energy between the first separate unit (8) and the second separate unit (8a).

8. Device (1) according to claim 7, wherein the interface for the transfer of data and/or energy between the first separate unit (8) and the second separate unit (8a) is provided in the form of a mechanical connection.

9. Device (1) according to claim 7 or 8, wherein the connecting means (11, 11a) comprise a plug connection.

10. Device (1) according to any one of the preceding claims, wherein the means for sensing (6) of a suitable position of the dispensing device (4) in relation to the eye (2) for wetting comprises a camera.

11. Device (1) according to any one of claims 4 to 10, wherein the sensing unit (8a) comprises a screen.

12. Device according to claims 10 or 11, wherein in the connected state the dispensing device (4) is located on the same side of the device (1) as the means for sensing (6) of a suitable position of the dispensing device (4) in relation to the eye (2) for wetting and is oriented in the same direction as the camera.

13. Device (1) according to any one of the preceding claims, wherein the means for sensing (6) of a suitable position of the dispensing device (4) in relation to the eye (2) for wetting is provided by a portable mobile device.

14. Device (1) according to claim 13, wherein the portable mobile device is selected from the group consisting of tablet computers, netbooks, laptops, digital media players, handheld personal computers, handheld game consoles, mobile phones, phablets, smartwatches and others.

15. Device (1) according to claim 13 or14, wherein the portable mobile device is a mobile telephone.

16. Device (1) according to any one of claims 2 to 15, wherein the first separate unit (8) does not comprise at least one of an energy storage and a data storage.

17. Device (1) according to any one of the preceding claims, comprising means for emitting of an acoustic signal when a suitable position for wetting has been sensed.

18. Device (1) according to any one of the preceding claims, comprising means for emitting of an optical signal when a suitable position for wetting has been sensed.

19. Device (1) according to any one of claims 11 to 18, wherein the device (1) is configured to depict in the region of the screen (7) a marking which, upon fixation with the eye (2) of the user, results in a suitable position for wetting.

20. Device (1) according to claim 20, wherein the marking is actively depictable on the screen (7).

21. Device (1) according to claim 20, wherein the marking is passively depictable on the screen (7) by a mirror image of the eye (2), and the device (1) is configured to actively depict on the screen (7) an auxiliary marking which, at a suitable position, correlates with the mirror image of the eye (2).

22. Device (1) according to any one of the preceding claims, further configured to measure the distance from the eye (2) and/or comprising position sensors by means of which the orientation of the device (1) is sensible.

23. Device (1) according to any one of the preceding claims, configured to output an optical and/or acoustic signal to guide the user to the suitable position.

24. Device (1) according to any of the preceding claims, configured to automatically actuate the triggering device (5) when a suitable position of the dispensing device (4) in relation to the eye (2) for wetting has been sensed.

25. Dispensing unit (8) comprising a reservoir (3), a dispensing device (4) and a triggering device (5) adapted to be coupled to a means for sensing (6) of a suitable position of the dispensing device (4) in relation to the eye (2) for wetting for a device (1) according to any one of the preceding claims.

26. Method for detecting a suitable position of a dispensing device for wetting an eye of a subject with a liquid (F), wherein the method is carried out using the device according to any one of claims 1 to 24, the method comprising the following step:
continuously detecting the position of a dispensing device (4) designed to withdraw a liquid (F) from a reservoir (3) and to use it for identification of a suitable position for wetting the eye (2);
wherein a signal is transmitted when a suitable position has been sensed using a means for sensing (6) of a suitable position of the dispensing device (4) in relation to the eye for wetting.

27. Method according to claim 26, wherein the signal is an acoustic and/or an optical signal.

28. Method according to claims 26 or 27, wherein a marking which, upon fixation with the eye (2) of the user, results in a suitable position for wetting is depicted on the screen (7).

29. Method according to claim 28, wherein indicators of a graphical nature that guide the user in the direction of the suitable position are depicted on the screen (7).

30. Method according to any one of claims 26 to 29, wherein the signal generated at the moment the suitable position for wetting has been reached is passed on to the triggering device (5), which on its part triggers the dispensing device (4).

31. Method according to any one of claims 26 to 30, wherein the signal generated at the moment the suitable position for wetting has been reached indicates to the user to manually actuate the triggering device (5).

32. Computer implemented method, causing a device (1) according to any of claims 1 to 24 to carry out the following steps when executed as part of a method according to any of claims 26 to 31:
(i) processing the image of an eye (2) of a subject captured by the means for sensing (6),
(ii) sensing a suitable position of the dispensing device (4) in relation to the eye (2) for wetting, and
(iii) transmitting the signal when a suitable position has been sensed.

33. Computer implemented method according to claim 32, further designed to identify the person using the device, to retrieve data saved in a data memory with respect to the treatment of said person, and to adjust the nature and/or number of signals for activation.
